(19) United States
(12) Patent Application Publication
(10) Pub. No.: US 2005/0008614 A1
(43) Pub. Date: Jan. 13, 2005

(54) Topical use of cytokines and chemokines for the treatment of viral or mykotic skin diseases or tumoral diseases

(76) Inventors: John Nieland, Stockdorf (DE); Christoph Rehfuess, Munchen (DE)

Correspondence Address: CLARK & ELBING LLP, 101 FEDERAL STREET, BOSTON, MA, 02110, US

(21) Appl. No.: 10/493,884

(22) PCT Filed: Nov. 7, 2002
(86) PCT No.: PCT/EP02/12438

(30) Foreign Application Priority Data
Nov. 7, 2001 (DE) 101 54 579.7

(51) Int. Cl. A61K 38/19

(52) U.S. Cl. 424/85.1

(57) ABSTRACT

The invention relates to the use of at least one cytokine and/or chemokine in the production of a topically acting medicament for treating viral and/or mykotic skin diseases and/or tumoral diseases.

The present invention relates to the topical use of at least one cytokine and/or chemokine for treating viral and/or mycotic skin diseases and/or tumor diseases and to a topically acting pharmaceutical formulation and its production.

The cytokine granulocyte-macrophage colony stimulating factor (GM-CSF) was originally isolated as a factor which stimulated the growth of macrophage- or granulocyte-containing colonies. GM-CSF is required for the growth and development of the precursor cells of granulocytes and macrophages. It stimulates myeloblasts and monoblasts and initiates the irreversible differentiation of these cells. It supports erythropoietin (EPO) in connection with the proliferation of precursor cells of erythocytes and megakaryocytes. Furthermore, GM-CSF is able to attract neutrophils. It augments the antimicrobial activities, oxidative metabolism and phagocytotic activities of neutrophils and macrophages. It also augments the cytotoxicity of these cells. Since GM-CSF is produced by cells (T lymphocytes, tissue macrophages, endothelial cells and mast cells) which are present at sites of inflammatory reactions, it can be assumed that GM-CSF is an important mediator of inflammatory reactions.

GM-CSF also exerts an influence on the function of the Langerhans cells of the skin. These cells are unable to induce a primary immune reaction. However, GM-CSF, together with IL4, converts them into highly active immunostimulatory dendritic cells.

GM-CSF acts synergistically with other cytokines, including the interleukins IL1, IL3 and IL4 and granulocyte-stimulating factor (G-CSF).

GM-CSF is used clinically for physiologically reconstituting hematopoiesis in the case of all diseases which are accompanied by inadequate maturation of blood cells or by a reduced production of leukocytes. What is probably the most important clinical use of GM-CSF is in the treatment of life-threatening neutropenia following chemotherapy and/or radiation therapy. GM-CSF can also be used to treat chemotherapy-induced cytopenias and cytopenia-dependent predispositions to infectious diseases and hemorrhages. In these cases, GM-CSF is normally administered in a dose of from 5 to 10 μg/kg/day, either by means of an intravenous infusion lasting from 4 to 6 hours or by means of subcutaneous injection.

In addition, GM-CSF is used for reconstituting the hematopoetic system following autologous or allogenic bone marrow transplantations. In this case, the therapeutic effect is based not only on reducing the duration of an absolute neutropenia but also on patients contracting significantly fewer infections, receiving fewer intravenous administrations of antibiotic and being hospitalized for a shorter period.

At present, cytokines such as GM-CSF or IL2, IL12 or IL18, and also the interferons IFNγ and IFNα, are being used or tested in a variety of indications in the field of tumor diseases. Thus, GM-CSF is being used in different ways, in combination with antigens, for stimulating the immune system, for example in the form of a GM-CSF-expressing tumor cell or in the form of a preceding or simultaneous administration of peptide antigens, for example derived from tumor antigens. These therapeutic forms are based on GM-CSF functioning as a type of adjuvant for an antigen which is administered in parallel (Lawson D, Kirkwood J M (2000) Granulocyte-macrophage colony-stimulating factor: another cytokine with adjuvant therapeutic benefit in melanoma J Clin Oncol 18(8):1603-5).

The object of the present invention was consequently to find further uses for cytokines.

It has now been found, surprisingly, that the topical use of cytokines and/or chemokines, such as GM-CSF, is suitable for treating viral and/or mycotic skin diseases as well as tumor diseases.

The present invention consequently relates to the use of at least one cytokine and/or chemokine for producing a topical pharmaceutical for treating viral and/or mycotic skin diseases and/or tumor diseases.

Because of the observed synergistic effects between different cytokines and/or chemokines, it is particularly advantageous if two, three, or even more than three, cytokines and/or chemokines are assimmilated simultaneously or in a chronologically staggered manner, with use being made, in particular, of three or, especially, two cytokines and/or chemokines. Particularly preferred combinations are GM-CSF, RANTES or MIP1α with IL4, IL2, IL12, IFNγ or TNFα.

In another preferred embodiment, the pharmaceutical does not comprise any constituents, such as tumor antigens or viral antigens, which essentially have an antigen effect. Constituents having an antigen effect are understood as meaning antigens or parts thereof which can induce an immune response in a patient. Within the meaning of the present invention, the term “essentially no constituents having an antigen effect” is understood as meaning constituents which have such a slight antigen effect that the possibility of the patient having an immune reaction which endangers the treatment can in general be ruled out.

The term “cytokine” is a universally applied designation for a large group of soluble proteins and peptides which function as humoral regulators, preferably in nanomolar to picomolar concentrations. These proteins and peptides modulate the functional activities of individual cells or tissues under normal or pathological conditions. They furthermore directly mediate interactions between cells and regulate processes which preferentially take place in the extracellular environment. “Chemokines” are a subgroup of the cytokines. They are relatively small proteins which, inter alia, act chemotactically on cells.

The term cytokines includes, for example, GM-CSF, G-CSF, IL1, IL2, IL3, IL4, IL5, IL6, IL7, IL8 IL9, IL10, IL11, IL12, IL13, IL14, IL15, IL16, IL17, IL18, IL19, IL20, IL21, IL22, IFNα, IFNβ, IFNγ, Flt3 L, Flt3 and TNFα. GM-CSF is a particularly preferred cytokine. The term chemokines includes, for example, RANTES, MIP1α, MIP1β, MIP1γ, MIP1δ, MIP2, MIP2α, MIP2β, MIP3α, MIP3β, MIP4, MIP5, MCP1, MCP1β, MCP2, MCP3, MCP4, MCP5, MCP6, 6cykine, Dcck1 and DCDF. GM-CSF, RANTES and MIP1α are particularly preferred chemokines. (With regard to the cytokines and chemokines: Cytokines Online Pathfinder Encyclopedia, Horst Ibelgauft's Hypertext Information Universe of Cytokines, Version 4.0, August 1999; Website: http://www.copewithcytokines.de/cope.cgi). Furthermore, within the context of this invention, the terms cytokine and chemokine are also understood as meaning variants of these cytokines and chemokines and also fusion proteins containing these cytokines and chemokines. This thereby encompasses, for example, point mutations, insertions and deletions as well as fusions with other polypeptides, for example with what are termed tags, for example His tag, GST tag, Myc tag or GFP tag, or fusions with functional domains of other polypeptides, for example with domains of immunoglobulins, or other cytokines or chemokines.

“Topical use” is understood as meaning the external application of the active compound on the skin. Preferably, the active compound is present in the form of an ointment, of a gel, of a plaster or of another skin-compatible formulation. The active compound is preferably applied locally in the region in which there is a skin change and/or a skin disease.

Viral skin diseases are understood as meaning skin diseases which are induced or caused by viruses and/or associated with viral infections. These include, for example, skin diseases such as skin warts, genital warts, anogenital warts or mucosal warts (see Table 1) which are elicited by at least one papillomavirus, in particular human papillomaviruses, such as HPV1, 2, 3, 4, 5, 6, 8, 9, 11, 12, 13, 14, 15, 16, 17, 18, 19, 20, 21, 22, 23, 24, 25, 26, 27, 28, 29, 31, 32, 34, 36, 37, 38, 46, 47, 48, 49, 50, 56 and 58. They also include diseases which are elicited by at least one herpesvirus, in particular by herpes simplex virus 1, herpes simplex virus 2, human herpesvirus (HHV) 1, HHV2, HHV3, HHV4, HHV7 and HHV8, as well as varicella zoster, that is herpes labialis, Kaposi's sarcoma, varicella and/or shingles.

TABLE 1

| Tumor | HPV types which induce it |
|---|---|
| skin virus warts | |
| verrucae plantares | 1, 2, 4 |
| verrucae vulgares | 2, 4 |
| verrucae plane juveniles | 3, 10 |
| epidermodysplasia | 5, 8, 9, 12, 14, 15, 17, |
| verruciformis | 19-29, 36-38, 46-50 |
| Anogenital warts | |
| condylomata acuminata | 6, 11 |
| condylomata plana | 6, 16, 31 |
| bowenoid papulosis | 16 |
| Mucosal warts | |
| papillomas on larynx and the | 6, 11 |
| oral mucosa | |
| focal epithelial hyperplasia | 13, 32 |

Tumor diseases which can be treated by the topical application of GM-CSF, for example, are tumors of the skin, for example melanoma or acute keratinosis, neoplasias of the genital and anal tract, such as cervical intraepithelial neoplasias, anal intraepithelial neoplasias or penile intraepithelial neoplasias (see Table 2 as well).

TABLE 2

| Tumor | HPV types which induce it |
|---|---|
| Malignant tumors | |
| Bowen's disease | rarely 2, 16, 34 |
| penile, anal and vulval | 6, 16, 18 |
| carcinoma | |
| cervical carcinoma | 16, 18 |
| laryngeal carcinoma | rarely 16, 18, 30 |
| lingual carcinoma | rarely 2, 16 |

Mycotic skin diseases are understood as meaning skin diseases which are caused by fungal infections. These include diseases of the skin, of the skin appendages and of the mucosae, including the external and internal genitals. They are understood, in particular, as being dermatomycoses (see Table 3) and other mycoses which can give rise to mycotic skin diseases (see Table 4 and Table 5).

TABLE 3
Mycoses
Important dermatomycoses

| Pathogen | Disease |
|---|---|
| Candida albicans | candidosis of the skin and |
| | skin appendages |
| Epidermophyton floccosum | tinea (T.) manuum et pedum, |
| | T. corporis, T. inguinalis, |
| | T. unguium |
| Exophilia werneckii | tinea nigra |
| Microsporum species | microsporia |
| Piedraia hortai | black piedra |
| Malassezia furfur | pityriasis versicolor |
| Scopulariopsis brevicaulis | T. unguium |
| Trichophyton mentagrophytes | trichophytosis, T. manuum |
| Trichophyton rubrum | et pedum, T. corporis, T. |
| other trichophyton species | inguinalis, T. |
| | granulomatosa nodularis, T. |
| | unguium |
| Trichophyton schoenleinii | favus |
| Trichophyton verrucosum | deep trichophytosis, T. |
| | capitis, T. barae |
| Trichosporon cutaneum | white piedra |

TABLE 4
Mycoses
Important cutaneous and subcutaneous (e.g. arising
posttraumatically) mycoses

| Pathogen | Disease | Manifestation |
|---|---|---|
| Cladosporium carrionii | chromoblastomycosis | skin, lymphatic |
| Phialophora compacta | | system, |
| Phialophora dematitidis | | generalization |
| Phialophora pedrosoi | | possible |
| Phialophora verrucosa | | |
| Sporothrix schenckii | sporothrix mycosis | |
| Cephalosporium species | eumycetoma | skin, lymphatic |
| Madurella grisea | (Mycetoma | system, skeletal |
| Madurella mycetomi | pedis) | system |
| Petriellidium boydii | | |

TABLE 5
Mycoses
Important pathogens of systemic mycoses and their
manifestation

| Pathogen | Disease | Manifestation |
|---|---|---|
| Opportunistic pathogens | | |
| Aspergillus fumigatus | Aspergillus | respiratory organs, |
| | mycoses | ear, generalization ear |
| | aspergilloma | |
| Aspergillus niger and | | Ear |
| others | | |
| Opportunistic pathogens | | |
| Candida albicans | Candida | gastrointestinal tract, |
| Candida glabrata | mycosis | respiratory organs, |
| Candida guilliermondii | | CNS, generalization |
| Candida krusei | | |
| Candida parapsilosis | | |
| Candida pseudotropicalis | | |
| Candida stellatoidea | | |
| Candida tropicalis | | |
| Cryptococcus neoformans | Cryptococcus | respiratory organs, |
| | mycosis | CNS, generalization |
| Rhizopus oryzae | Mucor mycosis | |
| Primary pathogenic | | |
| dimorphic fungi | | |
| Blastomyces | Blastomyces | respiratory organs, |
| dermatitidis | mycosis | skin, genitals, |
| Coccidioides immitis | Coccidioides | generalization |
| | mycosis | |
| Histoplasma capsulatum | Histoplasma | genitals, lymphatic |
| | mycosis | system, |
| Paracoccidioides | Paracoccidioides | generalization |
| brasiliensis | mycosis | |

According to the present invention, the pharmaceutical is in general produced by bringing at least one cytokine and/or chemokine into contact, for example by means of mixing or dissolution, with at least one suitable additive and/or auxiliary substance. The present invention therefore also relates to a process for producing a topically acting pharmaceutical, in which process at least one cytokine and/or chemokine is brought into contact with at least one auxiliary substance, in particular with human serum albumin, CpG or oxidized glutathione. The cytokine and/or chemokine is preferably prepared recombinantly, using methods known to the skilled worker.

Thus, it is possible, for example, to use an expression construct which is appropriate for recombinantly preparing the cytokine and/or chemokine and which contains a nucleic acid encoding the cytokine and/or chemokine as well as a promoter which is suitable for expression in, preferably, bacteria, e.g. E. coli, or in eukaryotic cells, such as in yeasts, e.g.. S. cerevisiae or S. pombe or Pichia pastoris, or in higher eukaryotic cells, for example in insect cells or human cells. Subsequently, the cytokine and/or chemokine can be purified under native or nonnative conditions, using methods known to the skilled person.

Methods which are customary in industrial pharmaceutics are employed to produce pharmaceuticals having a content of at least one cytokine and/or chemokine and to use these pharmaceuticals in the application according to the invention. For this, the active compounds are processed, preferably together with suitable pharmaceutically acceptable auxiliary substances and carrier substances, into the medicinal forms which are suitable for the various indications and sites of application. In this connection, the pharmaceuticals can be produced such that the release rate which is in each case desired, for example a rapid flooding and/or a retardation or slow-release effect, is achieved.

Customary emulsions, gels, ointments, creams of the mixed-phase or amphiphilic emulsion systems (oil/water-water/oil mixed phase), and also liposomes and transfersomes, or plasters, preferably ointments and creams, particularly preferably an ointment, may be cited as examples for conventional application to the skin or mucosa. The active compound is preferably applied locally in the region in which there is a skin or mucosal change and/or disease.

Additional topically applicable forms which can be produced are pastes, powders and solutions. As consistency-imparting bases, the pastes frequently comprise hydrophobic and hydrophilic auxiliary substances, preferably, however, hydrophobic auxiliary substances having a very high solids content. In order to increase their dispersity and flowability and glidability, and also to prevent agglomerates, the powders or topically applicable powders can also contain, for example, starch types, such as wheat starch or rice starch, flame-disperse silicon dioxide or silicaceous earths, which also serve as diluents.

In addition to the known uses on the skin and/or mucosa, the following special preparations are preferably suitable for use as pharmaceuticals which can be administered topically, locally or regionally: emulsions, creams, ointments, foam tablets or suppositories which can be applied genitally, vaginally or rectally, in particular genitally and vaginally. Rectal capsules can also be produced on the basis of gelatin or other carrier substances. Examples of suitable suppository bases are hydrogenated fats, such as Witepsol®, Massa Estarium®, Novata®, coconut butter, glycerol/gelatin compositions, glycerol/saponaceous gels and polyethylene glycols.

The medicinal forms which are in each case suitable can be produced in conformity with the formulation specifications and procedures, based on pharmaceutical/physical principles, which are known to the skilled person.

Examples of suitable auxiliary substances and/or carrier substances are sodium alginate, as a gelatinizing agent for producing a suitable base, or cellulose derivatives, such as guar gum or xanthan gum, inorganic gelatinizing agents, such as aluminum hydroxides or bentonites (what are termed thixotropic gelatinizing agents), polyacrylic acid derivatives, such as Carbopol®, polyvinylpyrrolidone, microcrystalline cellulose or carboxymethylcellulose. In addition, amphiphilic low molecular weight and higher molecular weight compounds, and also phospholipids, are suitable. The gels can be present either as water-based hydrogels or as hydrophobic organogels, for example based on mixtures of low molecular weight and high molecular weight paraffin hydrocarbons and vaseline. The hydrophilic organogels can, for example, be prepared on the basis of high molecular weight polyethylene glycol. These gelatinous forms can be washed off. However, the organogels which are preferred are the hydrophobic organogels. Particular preference is given to hydrophobic auxiliary substances and additives such as petroleum jelly, wax, oleyl alcohol, propylene glycol monostearate and propylene glycol monopalmitostearate. Furthermore, it is possible to add dyes, for example yellow and/or red iron oxide and/or titanium dioxide, for the purpose of color adjustment.

Examples of emulsifying agents which can be used are anionic, cationic or neutral surfactants, for example alkali soaps, metal soaps, amine soaps, sulfurized and sulfonated compounds, invert soaps, high fatty alcohols, partial fatty acid esters of sorbitan and polyoxyethylene sorbitan, e.g. lanette types, wool fat, lanolin and other synthetic products for producing oil/water and/or water/oil emulsions. Other examples of suitable auxiliary substances are ionic or anionic detergents, such as Triton X-100, Tween, sodium deoxycholate, and also polyols, such as polyethylene glycol or glycerol, sugars, for example sucrose or glucose, lipopolysaccharides, zwitterionic compounds, such as amino acids, such as glycine or, in particular, taurine or betaine, or lipids.

Vaseline, natural or synthetic waxes, fatty acids, fatty alcohols, fatty acid esters, for example as monoglycerides, diglycerides or triglycerides, paraffin or vegetable oils, hydrogenated castor oil or coconut oil, lard, synthetic fats, for example based on caprylic acid, capric acid, lauric acid and stearic acid, such as Softisan®, or triglyceride mixtures, such as Miglyol®, can be used as lipids in the form of fatty and/or oily and/or waxy components for producing the ointments, creams or emulsions.

In order to adjust the pH of the formulation, it is possible, for example, to use suitable organic or inorganic buffers, osmotically active acids and lyes, for example hydrochloric acid, citric acid, sodium hydroxide solution, potassium hydroxide solution or sodium hydrogen carbonate, and, in addition, buffer systems, such as citrate, phosphate buffer, Tris buffer (tris(hydroxymethyl)aminomethane, HEPES buffer ([4-(2-hydroxyethyl)piperazino]ethanesulfonic acid), MOPS buffer (3-morpholino-1-propanesulfonic acid) or triethanolamine. In general, the choice of the buffer depends on the buffer molarity which is desired.

In order to increase the stability, it is furthermore possible to add preservatives, such as methyl benzoate or propyl benzoate (parabene), sorbic acid, proteins, for example bovine, human or synthetic serum albumin, and/or protease inhibitors, such as aprotinin, ε-aminocarpoic acid, pepstatin A, EDTA or EGTA.

Auxiliary substances can also be penetration amplifiers, for example hydrophobic esters, such as isopropyl laureate, isopropyl myristate, isopropyl palmitate, isopropyl stearate, ethyl myristate, propyl myristate, butyl myristate and/or. ethyl oleate, in particular isopropyl myristate. In this connection, the term “hydrophobic” is understood as referring to compounds whose solubility in water is at most approx. 0.2 mg/ml, in particular at most approx. 0.1 mg/ml.

Other suitable auxiliary substances are adjuvants which amplify the immunogenic (sensitizing) effect of an antigen. In this regard, it is to be noted that the antigen against which an immunogenic effect is to be achieved is, in particular, a viral, mycotic and/or tumor-specific antigen which is expressed in the skin at the focus of the disease. Suitable adjuvants are, in particular, adjuvants which activate the toll-like receptors, for example adjuvants such as CpG oligonucleotides (Chen Y et al. (2001) Int. Immunol. 13, 1013-20), lipopolysaccharides (Alexander C and Rietschel E. T. (2001) J. Endotoxin Res. 7, 3, 167-202), Calmette-Guerin bacillus cell wall skeleton (Matsumoto M et al (2001) Int. Immunopharmacol. 1, 8, 1559-69) and also superantigens (Osanto S et al (2001) Infect. Immun. 69, 11, 6633-42) and agents which inhibit the signal effect of CTLA-4 (WO 00/32231, WO 97/20574). Examples of other suitable adjuvants are Freund's adjuvant, aluminum hydroxide, oxidized glutathione, double-stranded RNA and Iscom.

Preference is also given to using, as auxiliary substances, substances which stimulate the skin locally since this thereby increases the effect of cytokines and/or chemokines. DMSO is one of these substances, for example.

According to the present invention, the pharmaceutical can also be present as a formulation which comprises at least one cytokine and/or chemokine and a buffer solution or salt solution and/or an ointment base and also, where appropriate, at least one suitable additive and/or auxiliary substance.

The term formulation is understood as meaning a composition in the form of a solution or a suspension of said cytokines and/or chemokines, with the formulation in this connection essentially being a homogeneous formulation in which no sedimentation of ingredients can be observed even when the formulation is stored for a relatively long period at approx. 4° C.

Particular preference is given to a formulation in which the cytokine(s) and/or chemokine(s) which is/are present remain(s) native and/or active. The activity of the proteins can be demonstrated, in the case of each individual protein, in an appropriate activity test.

The salt, as an exemplary constituent of a formulation, is generally an alkali metal salt or alkaline earth metal salt, preferably a halide or phosphate, in particular an alkali metal halide, especially NaCl and/or KCl. The use of NaCl is particularly preferred.

Examples of preferred ointment bases are water-in-oil ointments, for example a wool fat alcohol ointment, comprising cetylstearyl alcohol, wool fat alcohol and vaseline or oil-in-water ointments (hydrophilic ointments), for example comprising emulsifying cetylstearyl alcohol, subliquid paraffin oil and vaseline.

Particular preference is given to using a previously described formulation in combination with a means for an occlusion, i.e. to the treated site being covered after the formulation has been applied to the skin, mucosa and skin appendages. This can be effected, for example, by means of a wound covering in the form of a plaster or a spray dressing.

Consequently, the present invention also relates to a treatment kit which comprises at least one cytokine and/or chemokine and also an occlusion means.

The figures and the following examples are intended to clarify the invention and other preferred embodiments and features of the invention without restricting them.

### DESCRIPTION OF THE FIGURES

FIG. 1 shows the graphic analysis of a skin penetration experiment carried out under 3 GM-CSF formulations in a flow-through diffusion cell. The distribution of the applied [¹²⁵I]-GM-CSF (in % of the total activity) in the receptor liquid (permeation), in the skin and in the ointment residues after 36 h is plotted against the formulation.

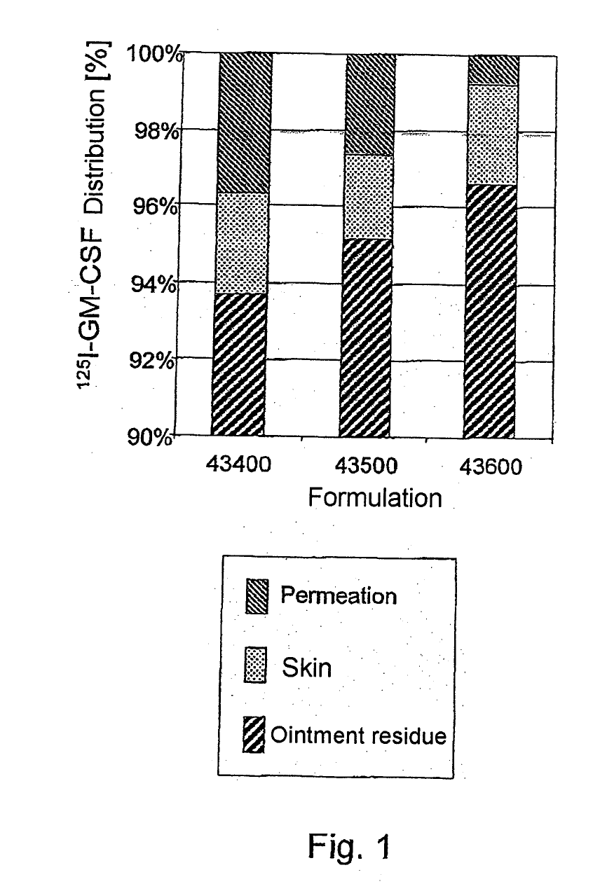

FIG. 2 shows the graphic analysis of a stability experiment carried out on formulations 43600 and 43400, in comparison with GM-CSF, at time 0 and after incubating at 40° C. for 7 days, with increasing quantities of GM-CSF being added to a constant quantity of DMSO ointment extract (X axis, logarithmically in % for ointment extracts containing GM-CSF and in units/ml for added GM-CSF) and the activation of monocytes being measured by their expression of CDla (Y axis).

FIG. 3 shows the graphic analysis of a stability experiment carried out on formulation 10101 at time 0 and after incubating at 40° C. for 7 days, with increasing quantities of GM-CSF being added to a constant quantity of DMSO ointment extract (X axis, logarithmically in %) and the activation of monocytes being measured by their expression of CDla (Y axis).

### EXAMPLE 1

### GM-CSF-Containing Formulations

1. Description of the Formulations

The following formulation mixtures, containing in each case 100 μg of human recombinant GM-CSF (Leucomax 400 from Sandoz AG, Basel, Switzerland)/g, were prepared:

TABLE 6

| Name | Type | Ingredients |
|---|---|---|
| 43400 | w/o | wool fat alcohol ointment: |
| | | cetylstearyl alcohol (0.5%) |
| | | wool fat alcohol (5%) |
| | | white vaseline (93.5%): |
| | | 75% Leucomax 400 (=human GM-CSF) in |
| | | 70% glycerol 400 μg/g: 25% |
| 43500 | w/o + | wool fat alcohol ointment: |
| | DMSO | cetylstearyl alcohol (0.5%) |
| | | wool fat alcohol (6%) |
| | | white vaseline (93.5%): |
| | | 75% Leucomax 400 in DMSO, |
| | | 400 μg/g: 25% |
| 43600 | o/w | hydrophilic ointment: |
| | | emulsifying cetylstearyl alcohol |
| | | (30%) |
| | | subliquid paraffin oil (35%) |
| | | white vaseline (35%): |
| | | 75% Leucomax 400 in 70% glycerol |
| | | 400 μg/g: 25% |
| | | was also prepared alternatively with |
| | | 100, 50 or 10 μg/g of murine GM-CSF |
| 10101 | PEG | polyethylene glycol ointment |
| | | polyethylene glycol 300 (100 g) |
| | | polyethylene glycol 1500 (100 g) |

w/o = water in oil;
o/w = oil in water

Preparing the Ointments:

All the ointments were prepared under aseptic conditions (laminar flow, sterile gloves, mouth guard, etc.).

20 g of the ointment designated 43400 were prepared as follows:

Initially, for preparing the wool fat alcohol ointment, 1 g of cetylstearyl. alcohol, 12 g of wool fat alcohol and 187 g of white vaseline were weighed into a glass beaker and melted at 70° C. and then cooled down to room temperature while being stirred continuously. Leucomax 400 was dissolved in 1 g of 70% glycerol solution (5×400 μg/g receptacles=2000 μg/5 g). 15 g of wool fat alcohol ointment were then introduced into a mortar, after which 5 g of Leucomax/glycerol solution, 400 μg/g, were added and mixed in homogeneously. 1 g quantities of the ointment were in each case aliquoted into receptacles.

20 g of the ointment having the designation 43500 were prepared as follows:

Initially, for preparing the wool fat alcohol ointment, 1 g of cetylstearyl alcohol, 12 g of wool fat alcohol and 187 g of white vaseline were weighed into a glass beaker and melted at 70° C. and then cooled down to room temperature while being stirred continuously. Leucomax 400 was dissolved in 1 g of highly pure dimethyl sulfoxide (DMSO) (5×400 μg/g receptacles=2000 μg/5 g). 15 g of wool fat alcohol ointment were introduced into a mortar, after which 5 g of Leucomax/DMSO solution, 400 μg/g, were added and mixed in homogeneously. 1 g quantities of the ointment were in each case aliquoted into receptacles.

20 g of the ointment having the designation 34600 were prepared as follows:

Initially, 70 g of white vaseline, 70 g of subliquid paraffin oil and 60 g of emulsifier cetylstearyl alcohol were weighed into a glass beaker, melted at 70° C. and then cooled down to room temperature while being stirred continuously. Leucomax 400 was dissolved in 1 g of 70% glycerol solution (5×400 μg/g receptacles=2000 μg/5 g). 15 g of the hydrophilic ointment were introduced into a mortar, after which the Leucomax/glycerol solution, 400 μg/g, was added and mixed in homogeneously. 1 g quantities of the ointment were in each case aliquoted into receptacles.

20 g of the ointment having the designation 10101 were prepared as follows:

Initially, 100 g of polyethylene glycol 300 and 100 g of polyethylene glycol 1500 were melted at from 60 to 70° C., while being stirred, and then stirred until the mixture had cooled down. Leucomax 400 was dissolved in 1 g 70% glycerol solution (5×400 μg/g receptacles=2000 μg/5 g). 15 g of the polyethylene glycol mixture were introduced into a mortar, after which the Leucomax/glycerol solution, 400 μg/g, was added and mixed in homogeneously. 1 g quantities of the ointment were in each case aliquoted into receptacles.

2. Preparing Formulations Containing ¹²⁵I-Labeled Human GM-CSF

¹²⁵I-labeled human GM-CSF (NEN Life Sciences, Cologne, Germany, ¹²⁵I-PACAP27, NEX294, lot number GCB1500) was calibrated. At the beginning of the experiment, the specific activity was 5.2 μCi (192.4 kBq). The protein was obtained in lyophilized form and taken up in 70 μl of distilled water (a homogeneous solution was not obtained when it was taken up in 35 μl). The resulting specific activity of the solution was 0.074 μCi/μl (2.75 kBq/μl).

Approx. 260 mg of the three different GM-CSF formulations were transferred to a 48-well cell culture dish. 15 μl of ¹²⁵I-labeled GM-CSF were added and mixed with the formulation, at room temperature, using a spatula. Between 3 and 5 mg of the mixture were transferred to a scintillation vessel and 2 ml of Packard Microscint-20 were added. The samples were heated at 60° C. for several minutes and then mixed once again. After that, they were measured in a scintillation counter (see table 7). The mixed formulations were transferred to Eppendorf tubes and stored at 4° C.

As a control, 2 μl of the ¹²⁵I-GM-CSF solution (0.074 μCi/μl, 2.75 kBq/μl) were measured directly in 2 ml of Packard Microscint-20.

TABLE 7

| | | | | | | | % of |
|---|---|---|---|---|---|---|---|
| | | added | | | | | theo- |
| Formu- | | ¹²⁵I-GM- | | measured | | | retical |
| lation | Weight | CSF/μl | Bq/mg | mg | cpm | cpm/mg | activity |
| 43400 | 263 mg | 15 | 157 | 5 | 7.372 | 1.474 | 15.6 |
| | | | | 4 | 6.415 | 1.603 | 17.0 |
| 43500 | 254 mg | 15 | 162 | 4 | 5.778 | 1.444 | 14.8 |
| | | | | 3 | 5.119 | 1.706 | 17.6 |
| 43600 | 280 mg | 15 | 147 | 4 | 4.303 | 1.075 | 12.2 |
| | | | | 3 | 3.140 | 1.046 | 11.8 |
| Control | | 2 | | | 76.890 | | 23.0 |

On the basis of the control, it can be seen that this method can only be used to measure 23% of the activity which is to be expected theoretically.

This experimental series makes clear that it was possible to introduce an aqueous solution of GM-CSF (in this case present as ¹²⁵I-labeled GM-CSF) into all three formulations. It is possible to introduce ¹²⁵I-labeled GM-CSF homogeneously into the formulations described, as can be seen from the fact that the parallel analyses of the individual formulations only show slight differences. It was possible to detect, in the different formulations, between 11.8 and 17.6% of the quantity of GM-CSF which was in theory originally added, with the maximally detectable quantity, as determined by the system, being 23%.

### EXAMPLE 2

### Skin Penetration by Different GM-CSF Formulations

The ability of different GM-CSF formulations to penetrate the skin was measured by means of an in vitro skin penetration assay (see, for example: Bronaugh R L et al., (1982) Toxicol Appl Pharmacol 15;62(3): 481-488; Bronaugh R L, (2000) Ann N Y Acad Sci 919: 188-191).

Skin samples of female abdominal origin from cosmetic operations were used. Skin disks (450 μm) which contained the stratum corneum, the epidermis and part of the dermis, were prepared in a dermatome. Skin disks having a diameter of 10 mm were punched out and placed in a diffusion cell. The skin pieces were frozen between microscope slides at ≦−15° C. The thickness of the skin disks was measured between the slide mountings. Since preparation of the membrane could be accompanied by damage to the skin, the integrity of the skin membrane was checked microscopically before the membrane was fitted into the diffusion cell.

The diffusion cell employed was that provided by BSL BIOSERVICE, Planegg, Germany. This cell is designed with a PTFE donor and PTFE acceptor for flow-through diffusion for horizontal exposure of the skin surface. The area of the exposed skin is 19.63 mm². The temperature of the diffusion cells was kept constant. A multichannel peristaltic pump was connected to the receptor part of the diffusion cell and a programable fraction collector was used for collecting the samples.

The frozen skin was washed with PBS and placed on the acceptor part. The diffusion cell was closed with the receptor part and equilibrated (adjustment of flow rate and temperature to 0.6 ml/h and 32±1° C.) for at least 1 h, in horizontal position, with degassed acceptor liquid (physiological phosphate-buffered sodium chloride solution (RT, pH 7.5±0.3), supplemented with 0.1% BSA and 50 μg gentamycin/ml; degassed). After that, a defined quantity of the ¹²⁵I-labeled GM-CSF formulations mentioned in Example 1.2 was applied to the skin surface using a spatula. The collection of the acceptor liquid was started for 36, with the collecting vessel being changed every 6 h. At the end of the series of measurements, the remaining ointment was wiped off from the donor site using a cottonwool stick. After the diffusion cell had been disassembled, the skin membrane was wiped off once again with a cottonwool stick and all the remaining, unpenetrated ointment residues were collected in a scintillation vessel.

200 μl of each collected fraction were mixed with 2 ml of scintillation liquid (Microscint™20, Canberra Packard, Dreieich, Germany). The collected, unpenetrated ointment residues were mixed with 4 ml of scintillation liquid and incubated at 60° C., for at least 30 min, in an ultrasonication waterbath. The exposed skin disks were incubated with 500 μl of tissue dissolver (Solvable™, Packard) at 50° C. for at least 3 h. After cooling down to room temperature, 50 μl of the dissolved skin were added to 4 ml of scintillation liquid. The radioactivities of all these samples were measured in a scintillation counter (in cpm) and the distribution of the [¹²⁵I]-GM-CSF, expressed as activity (nCi), in the skin, in the unpenetrated ointment residues and in the acceptor liquid was determined in accordance with the formula.
Activity[nCi]=cpm×dilution factor×quench factor/60/37

P values were calculated using the unpaired t test. In the described experiment, it was demonstrated that between 1.4 and 3.7% of the applied [¹²⁵I]-GM-CSF penetrated through the exposed skin layers into the acceptor liquid within 36 h. In this connection, formulation 43600 was found to give the lowest penetration while 43400 gave the highest penetration. After the experiment had been concluded, 2.6% (43400), 2.2% (43500) and 3.5% (45600) of the applied quantity of [¹²⁵I]-GM-CSF was detected in the skin. If it is assumed that both the GM-CSF in the skin and the GM-CSF which has migrated through the skin is therapeutically active, >6% of the applied GM-CSF is active in the case of formulation 43400, while <5% is active in the case of formulation 43500 and <4% is active in the case of formulation 43600.

### EXAMPLE 3

### Stability of Different GM-CSF Formulations

In order to test the stability of different GM-CSF formulations, approx. 10 mg of the formulations mentioned in Example 1.1 were weighed out under sterile conditions and incubated at 40° C. for 7 days. 5 μl of DMSO/mg of cream were then added to the samples in order to extract the GM-CSF which was present in the cream. The samples were mixed thoroughly (vortexing) and incubated at RT for approx. 2 h, with the samples being mixed once again by vortexing approx every 30 min. The samples were centrifuged down briefly and stored at 4° C. until they were subjected to further processing.

Monocytes were isolated using known methods (see, for example, Current Protocols in Immunology, Editors Coligan J E et al. 1999, pages 7.32.1-4). They were then taken up in medium (RPMI—Gibco, Paisley, Scotland—containing 5% FCS) such that a concentration of 175000-220000 cells per 500 μl was obtained. To add human recombinant IL4 (Pharmingen, Heidelberg, Germany) at double concentration (final conc. 500 units/ml). The extracted ointment samples were equilibrated to RT; after 30 min, they were mixed thoroughly by vortexing and centrifuged down briefly.

In each case 1 ml of medium (RPMI containing 5% FCS) was initially introduced into each well of a 48-well plate after which 2.5 μl of the cream extracts or, as control, 0.5 μl of human recombinant GM-CSF (corresponds to 0.25% extract or 500 units of GM-CSF) were added. Dilution series were prepared from each of these starting mixtures by in each case pipetting 500 μl of what was in each case the more highly concentrated mixture into wells which in each case contained 500 μl of introduced medium. The appearance of this dilution series was then as depicted in Table 8.

TABLE 8

| | Extract/% | GM-CSF/units |
|---|---|---|
| Starting mixture: | 0.25 | 500 |
| 1^{st} dilution: | 0.125 | 125 |
| 2^{nd} dilution: | 0.06 | 60 |
| 3^{rd} dilution: | 0.03 | 30 |
| 4^{th} dilution: | 0.015 | 15 |
| 5^{th} dilution: | 0.0075 | 7.5 |
| 6^{th} dilution: | 0.0037 | 3.75 |

After that, 500 μl of the monocyte suspension were added to each well. The plates were incubated at 37° C. for 7 days after which the cells were harvested and stained with mouse anti-human CDla (1:50, Pharmingen, Heidelberg, Germany) and anti-mouse IgG FITC (3/50, Sigma, Deisenhofen, Germany). In conclusion, the CD1a immune staining of the cells was analyzed in a FACS (FACS Calibur, from Beckton Dickenson, Hamburg, Germany).

A mixture composed of 500 μl of medium and 2.5 μl of human recombinant GM-CSF was used as the positive control, while 500 μl of medium without GM-CSF was used as the negative control.

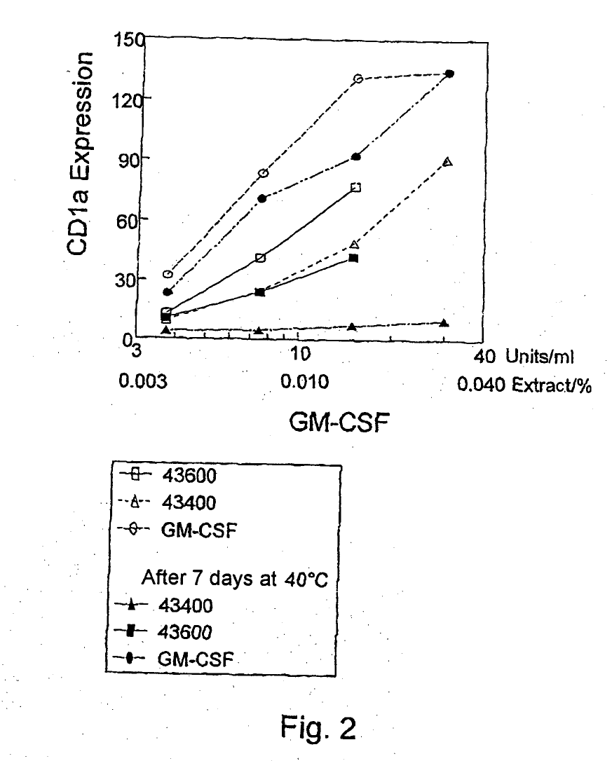

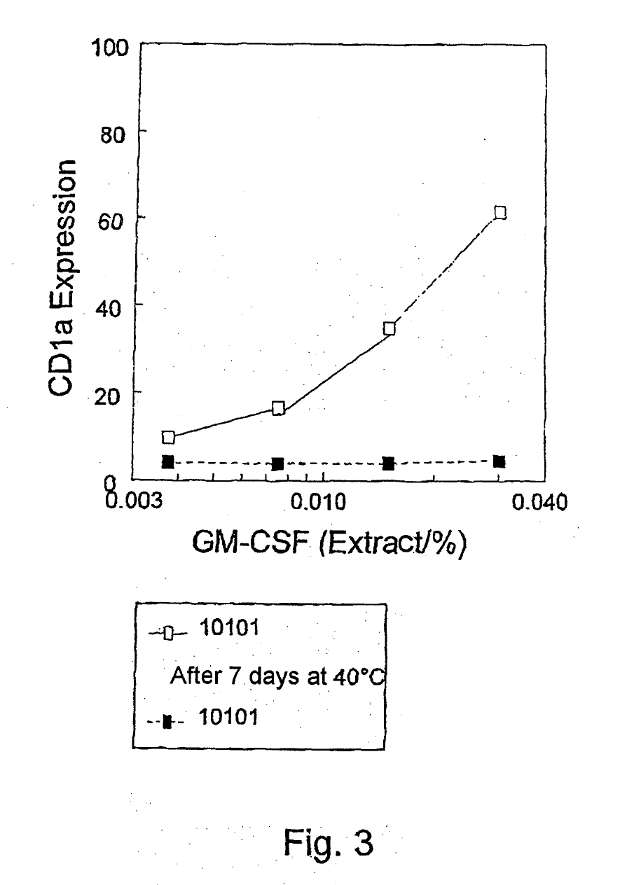

The stability experiments showed that, after 7 days at 40° C., formulation 43600 possessed markedly more GM-CSF activity than did formulation 43400 (see FIG. 2). Formulation 10101 hardly had any GM-CSF activity left after the incubation time, either (see FIG. 3).

1-13. (Cancelled)

14. A method of treating a condition selected from the group consisting of mycotic skin diseases, neoplasias of the genital tract and anal tract, Bowen's disease, laryngeal carcinoma, lingual carcinoma, and skin diseases caused by at least one virus selected from the group consisting of a papilloma virus and a herpes virus, said method comprising administering to a patient at least one agent selected from the group consisting of a cytokine and chemokine.

15. The method as claimed in claim 14, wherein more than three agents are administered.

16. The method as claimed in claim 14, wherein three agents are administered.

17. The method as claimed in claim 14, wherein two agents are administered.

18. The method as claimed in claim 14, wherein essentially no additional constituents acting as agents are administered.

19. The method as claimed in claim 14, wherein the agent is selected from the group consisting of GM-CSF, G-CSF, IL1, IL2, IL3, IL4, IL5, IL6, IL7, IL8, IL9, IL10, IL11, IL12, IL13, IL14, IL15, IL16, IL17, IL18, IL19, IL20, IL21, IL22, IFNα, IFNβ, IFNγ, Flt3 L, Flt3, RANTES, MIP1α, MIP1β, MIP1γ, MIP1δ, MIP2, MIP2α, MIP2β, MIP3α, MIP3β, MIP4, MIP5, MCP1, MCP1β, MCP2, MCP3, MCP4, MCP5, MCP6, 6cykine, Dcck1, and DCDF.

20. The method as claimed in claim 14, wherein the agent is selected from the group consisting of GM-CSF, RANTES, and MIP1α.

21. The method as claimed in claim 14, wherein the papilloma virus is a human papilloma virus.

22. The method as claimed in claim 14, wherein the papilloma virus is selected from the group consisting of HPV 1, 2, 3, 4, 5, 6, 8, 9, 11, 12, 13, 14, 15, 16, 17, 18, 19, 20, 2, 22, 23, 24, 25, 26, 27, 28, 29, 31, 32, 34, 36, 37, 38, 46, 47, 48, 49, 50, 56, and 58.

23. The method as claimed in claim 14, wherein the herpes virus is selected from the group consisting of herpes simplex virus 1, herpes simplex virus 2, varicella zoster virus, human herpes virus 1, 2, 3, 4, 7, and 8.

24. The method as claimed in claim 14, wherein the viral skin diseases are selected from the group consisting of warts, genital warts, benign tumors of the skin and mucosa caused by papilloma viruses.

25. The method as claimed in claim 14, wherein the viral skin diseases are selected from the group consisting of verrucae plantares, verrucae vulgares, verrucae planae juveniles, epidermodysplasia verruciforis, condylomata acuminata, condylomata plana, bowenoid papulosis, papillomas of the larynx, papillomas of the mucosa, focal epithelial hyperplasia, herpes labialis, Kaposi's sarcoma, varicella, and shingles.

26. The method as claimed in claim 14, wherein the neoplasias of the genital tract and anal tract are selected from the group consisting of penile carcinoma, anal carcinoma, vulval carcinoma, and cervical carcinoma.

27. The method as claimed in claim 14, wherein the mycotic skin diseases are selected from the group consisting of dermatomycoses, chromoblastomycosis, Sporothrix mycosis, eumycetoma, and systemic mycoses.

28. A method for producing a topically acting pharmaceutical, said method comprising bringing into contact at least one agent selected from the group consisting of a cytokine and a chemokine with at least one auxiliary substance.

29. The method as claimed in claim 28, wherein the auxiliary substance is selected from the group consisting of human serum albumin, CpG, and oxidized glutathione.

30. The method as claimed in claim 28, wherein the agent is prepared recombinantly.

31. A topically acting pharmaceutical formulation which comprises at least one agent selected from the group consisting of a cytokine and a chemokine, as well as cetylstearyl alcohol, vaseline and wool fat alcohol.

32. A topically acting pharmaceutical formulation which comprises at least one agent selected from the group consisting of a cytokine and a chemokine, as well as cetylstearyl alcohol, vaseline and paraffine oil.

33. A treatment kit which comprises a formulation as claimed in claim 31 or 32 and an occlusion means.

34. The treatment kit as claimed in claim 33, wherein the occlusion means is a plaster.

35. The treatment kit as claimed in claim 33, wherein the occlusion means is a spray dressing.
